(19) United States
(12) Patent Application Publication
(10) Pub. No.: US 2023/0200983 A1
(43) Pub. Date: Jun. 29, 2023

(54) Devices, Systems, and Methods for a Collapsible and Expandable Replacement Heart Valve

(71) Applicant: ReValve Solutions Inc., Bainbridge Island, WA (US)

(72) Inventors: Julie Logan Sands, Bainbridge Island, WA (US); Kenneth Eugene Perry, Bainbridge Island, WA (US); Edward Ian Wulfman, Bainbridge Isalnd, WA (US); Philip Andrew Ahrens, Bainbridge Island, WA (US)

(73) Assignee: ReValve Solutions Inc., Bainbridge Island, WA (US)

(21) Appl. No.: 17/925,590

(22) PCT Filed: May 17, 2021
(86) PCT No.: PCT/US21/32817

§ 371 Date: Nov. 15, 2022

Related U.S. Application Data
(60) Provisional application No. 63/025,881, filed on May 15, 2020.

(51) Int. Cl. A61F 2/24

(52) U.S. Cl. CPC A61F 2/2418; A61F 2/2439; A61F 2/2436; A61F 2220/0075

(57) ABSTRACT

Disclosed herein are systems and methods for a replacement heart-valve delivery system, comprising a replacement heart valve and a delivery component. The delivery component enables control of the replacement heart valve such that it can control collapsing and expanding the heart valve. Due to the nature of the flexible wire frame making up the heart valve, as well as its size, the delivery component allow for flexibility in the delivery catheter. The claimed invention thus allows for less traumatic delivery, more precise delivery, and a greater number of options on how to deliver.

### CROSS-REFERENCE TO RELATED APPLICATIONS

This application claims benefit to U.S. Provisional Application No. 63/025,881, filed on May 15, 2020, titled Devices, Systems, and Methods For a Collapsible and Expandable Replacement Heart Valve; the contents of all of which are incorporated herein by this reference as though set forth in their entirety, and to which priority and benefit are claimed.

### FIELD OF USE

The present disclosure relates generally to replacement heart-valve technology, and more specifically to devices, systems, and methods for a collapsible and expandable, heart-valve assembly that is highly flexible, resilient, retractable, and replaceable.

### BACKGROUND

Heart-valve intervention, such as full open-heart surgery, is often required to treat diseases of one or more of the four heart valves, which work together to keep blood properly flowing through the heart. Replacement and/or repair of a heart valve is often required when a valve is “leaky” (e.g., there is mitral valve regurgitation) or when a valve is narrowed and does not open properly (e.g., mitral valve stenosis). Typically, heart-valve replacement, such as mitral-valve replacement, involves replacement of the heart's original (native) valve with a replacement, mechanical and/or tissue (bioprosthetic) valve. Yet this results in problems with the replacement of valves and/or the frames carrying them, including: a) degradation of the leaflets (valve-like structure); b) breaking or failing frames, particularly with laser-cut nitinol frames; and c) undesirable changing in size of the native valve annulus. Replacement heart valves pose additional problems after they are implanted. For example, the replacement valve may move or migrate after it is placed in a desired location in the heart, or its location may not permit proper directional flow of blood during delivery. Replacement valves are also not readily retrievable, most often because such removal can damage the surrounding heart tissue. This can be particularly problematic, for example, if the replacement valve is not properly and accurately placed into position when it is implanted in the native heart, as well as when the replacement valve starts failing, which may occur years after initial implantation. An additional problem is that typical replacement valves, especially laser-cut valve frames, are relatively stiff and inflexible, resulting in a valve that does not flex with the dynamic movements of the pumping heart. Such inflexible valves do not conform to such dynamic movements, which can cause trauma to the heart surfaces, cause breaks in the frame itself, otherwise cause or exacerbate problems during or after implantation.

Additionally, although percutaneous implementation of prosthetic valves using a catheter is preferred—because it avoids traumatic open surgery and the transcatheter route via the aorta to the aortic valve is without much tortuosity—it too comes with challenges. For example, implanting prosthetic valves in other malfunctioning native valves (such as a vena cava-trans septal route to native mitral valves, or a vena cava route to the tricuspid valves) offers larger challenges in terms of tortuosity, as a catheter may need to turn 180 degrees or more near the delivery site.

Thus, what is needed are devices, systems, and methods for a replacement heart valve that enables compact and secure delivery into the heart and convenient control of expansion and retraction of the valve when being implanted or removed, preferably entirely via a catheter; and which ensures proper directional flow of blood through the heart during and after a valve replacement procedure. Also needed are improved devices, systems, and methods for transcatheter delivery of prosthetic valves.

### SUMMARY OF THE DISCLOSURE

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The present disclosure is directed to devices, systems, and method for a collapsible, replacement heart-valve assembly (referred throughout this disclosure as “valve assembly”) that is highly flexible, resilient, retractable, and replaceable. And is directed to devices, systems, and methods for delivery and placement of the heart-valve assembly. As disclosed herein, the valve assembly has the capability to be replaced years after implantation if problems, such as recurrent mitral valve regurgitation, arise.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1 generally illustrates an embodiment of a collapsible, heart-valve assembly system as disclosed herein.

FIG. 2 generally illustrates an embodiment of a tubular, braided frame as disclosed herein.

FIG. 3 generally illustrates an embodiment of a tubular, braided frame as disclosed herein.

FIG. 4 generally illustrates an embodiment of a tubular, braided frame as disclosed herein.

FIG. 5 generally illustrates an embodiment of a leaflet panel as disclosed herein.

FIG. 6 generally illustrates an embodiment of a Z-valve insert as disclosed herein.

FIGS. 7A-7G generally illustrate embodiments of patterns for leaflet panels.

FIG. 8 generally illustrates an embodiment of a collapsible, heart-valve assembly system as disclosed herein.

FIG. 9 generally illustrates an embodiment of a collapsible, heart-valve assembly system as disclosed herein.

FIG. 10A generally illustrates an embodiment of a collapsible, heart-valve assembly system as disclosed herein.

FIG. 10B generally illustrates an embodiment of a collapsible, heart-valve assembly system as disclosed herein.

FIG. 11 generally illustrates an embodiment of delivery system for a collapsible, heart-valve assembly as disclosed herein.

FIG. 12 generally illustrates an embodiment of a delivery system of a collapsible, heart-valve assembly as disclosed herein.

FIG. 13 generally illustrates an embodiment of a delivery system of a collapsible, heart-valve assembly as disclosed herein.

FIG. 14 generally illustrates an embodiment of a delivery system of a collapsible, heart-valve assembly as disclosed herein.

FIG. 15 generally illustrates an embodiment of a retrieval system of a collapsible, heart-valve assembly as disclosed herein.

FIG. 16 generally illustrates an embodiment of a braided frame of a collapsible, heart-valve assembly as disclosed herein.

FIG. 17 generally illustrates an embodiment of a deployment system for a collapsible, heart-valve assembly system as disclosed herein.

### DETAILED DESCRIPTION OF EMBODIMENTS

Before the present systems and methods are disclosed and described, it is to be understood that the systems and methods are not limited to specific methods, specific components, or to particular implementations. It is also to be understood that the terminology used herein is for the purpose of describing particular embodiments only and is not intended to be limiting. Various embodiments are described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

Disclosed herein is a collapsible heart-valve assembly system (the “valve assembly”) comprising at least a braided, collapsible frame and leaflet assembly that together serve to provide a sealing portion. The valve assembly may be delivered through a catheter and may perform as either a standalone valve replacement or placed with an existing receiver structure. The valve assembly may further comprise attachments and additional features for catheter delivery, positioning and partial deployment, and retrieval.

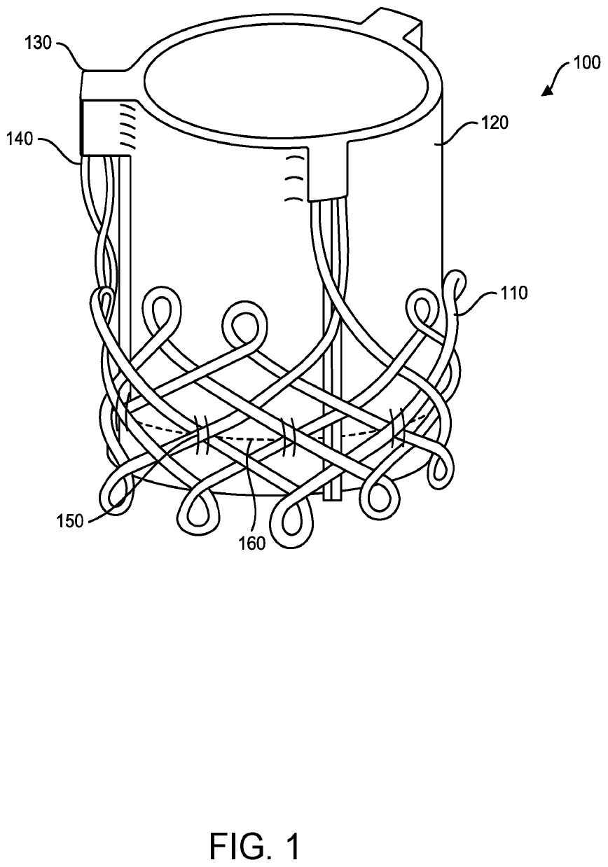

FIG. 1 generally illustrates an embodiment of a collapsible, heart-valve assembly system as disclosed herein. In a preferred embodiment, as shown in FIG. 1, a valve assembly 100 may comprise a tubular, braided frame 110 and a leaflet assembly 120 incorporated into the frame 110, with the frame 110 comprising one or more commissure posts 140. Commissure posts 140 may be defined as attachment points for the downstream portion of the leaflet assembly 120. In a preferred embodiment, a frame comprises three commissure posts, though it is common for a frame to have two or four commissure posts. Commissure posts 140 do not need to be symmetrically disposed around the valve, nor must they be the same height. And when compressed, the commissure posts can elastically deform to accommodate not deforming a non-flexible leaflet material.

The valve assembly 100 may further comprise one or more tabs 130, wherein the tabs 130 are sewn to the commissure posts 140. The valve assembly 100 may also comprise a base stitch 150 along a row of lashed crossing points of the frame 110, wherein the base stitch 150 connects the frame 110 to the leaflet assembly 120 along the circumference of the leaflet assembly's centerline 160. A base stitch 150 may be defined as the stitching line that delineates the inflow edge of a functional valve assembly 100.

The valve assembly disclosed herein is novel and an improvement over the prior art because it combines a minimal braided, wire structure with a novel leaflet-assembly design, wherein the integration of both together in a strategic manner provides a valve that may be compressed to a very low profile that is much smaller than other, existing percutaneously delivered valves. Additionally, the design of the valve assembly creates an option for ease of removal, either by percutaneous techniques or by minimally invasive surgical techniques. And the valve assembly may be delivered in a pre-placed receiver, such that the valve may have a minimal wire structure combined with the leaflet assembly and attachment strategy.

Benefits of this disclosure over prior art, include but are not limited to a less invasive and less traumatic puncture to accommodate delivery of the valve. Further, the profile of the valve and the strategic combination of wire braid frame, leaflet assembly, and attachment strategy allows for a more flexible delivery system. This is due to the nature of the flexible wire frame as well as because the valve is relatively short and has delivery system features that allow for flexibility in the delivery catheter. The combination of these factors allows for less traumatic delivery, more precise delivery, and a greater number of options on how to deliver.

Additionally, the ability to remove by percutaneous or minimally invasive techniques is a unique advantage of this valve assembly. Current state-of-the-art implanted valves, if they become malfunctioning or disabled, must either be removed by major surgical techniques or by implanting a second valve inside the malfunctioning valve. Both of these options have major drawbacks. Major surgery often is obviated by a patient because of age or physical condition. And implanting a valve inside an existing valve compromises the newly implanted valve and reduces the options if it does not work as intended.

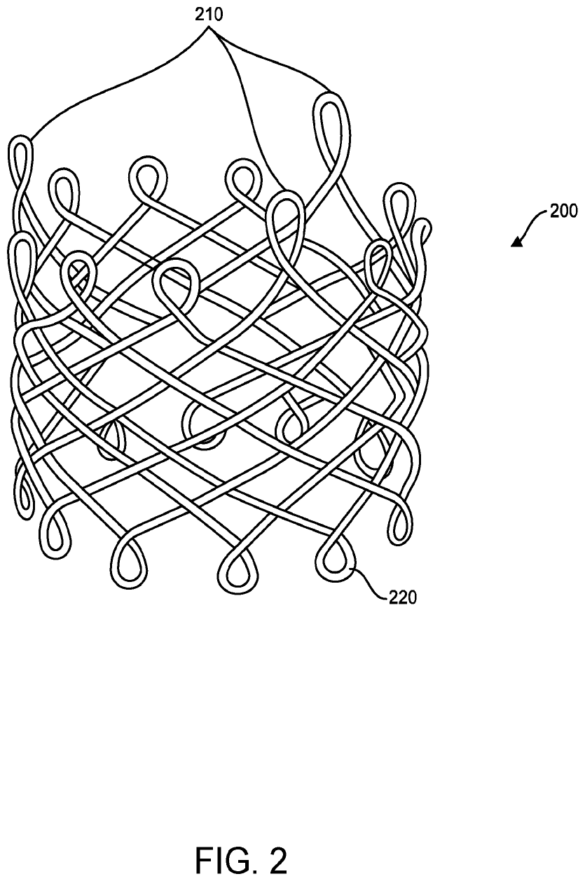

FIG. 2 generally illustrates an embodiment of a tubular, braided frame as disclosed herein. A braided, valve frame may be defined as a single or multi-wire, braided, self-expanding frame that supports the leaflets and provides a sealing portion. As shown in FIG. 2, the tubular frame 200—with corresponding length, diameter, distal end, and a proximal end—comprises one or more commissure posts 210 at the distal end and loops 220 at both distal and proximal ends. The commissure posts 210 typically extend out from the tubular frame 200 by a minimum amount, for example from 10% to 30% of the length of the frame 200. The loops 220 may be a simple 300-360-degree loop back of the material making up the frame 200, or may be more than 360 degrees, i.e. two turns of the frame material. The loops 220 provide a stable end to the frame 200, allow for means of looping sutures to a delivery mechanism, provide an attachment means for the leaflet structure 120, and reduce the peak stresses/strains at the turns. These features can be designed to enhance or reduce the radial force of the system. Additionally, the loops 220 may be used for positioning radiopaque markers for visibility under fluoroscopy.

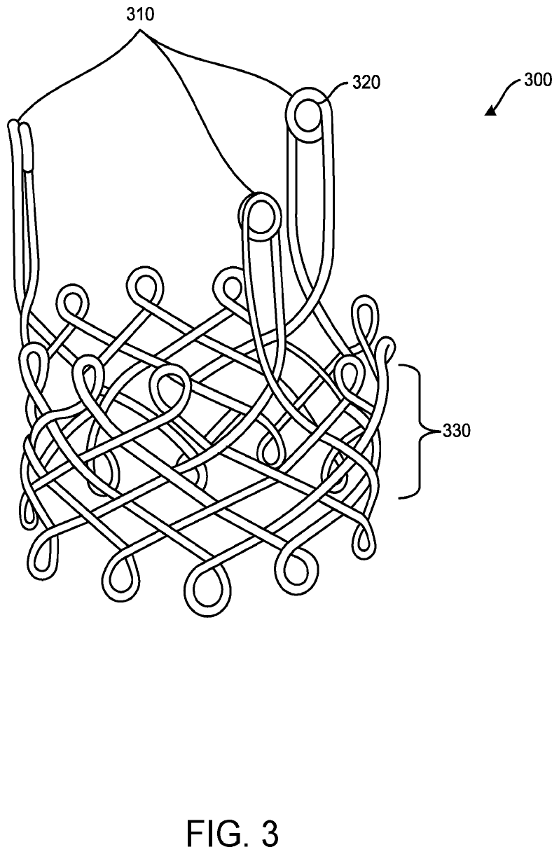

FIG. 3 generally illustrates an embodiment of a tubular, braided frame as disclosed herein. As shown in FIG. 3, a braided frame 300 may vary in size. For example, the commissure posts 310 may extend further from the distal end of the frame 300. In this embodiment, the commissure posts 310 have a loop 320 that is greater than 360 degrees, creating nearly two full loops. The center 330 of the main body of the frame 300 provides a stable centerline between distal and proximal ends of the frame 300, where a suture line can be made to sew the proximal end of the leaflet structure. An embodiment of this is braid lashing, defined as sutures used to constrain the motion of crossing points in the braid and may be horizontally and/or vertically oriented. The proximal end of the frame 300 may typically be used to provide structure to connect the leaflet structure in a 360-degree suture line and to create a sealing zone for valve function when the valve is inserted into a receiver. A sealing zone generally refers to the region on the outside of the frame between an inflow stitch (disclosed further down, see 540; an inflow stitch is generally parallel to and on the inflow side of the base stitch and serves to attach a leaflet assembly to a frame or cuff;) and base stitch (previously disclosed, see 150) so as to provide a larger area over which sealing occurs. Sealing, such as through the use of a sealing ring, refers to the prevention of blood flow from either side while the leaflets provide flow control. This centerline is stable because when the braided structure is compressed for delivery, the structure elongates and so the proximal and distal ends move away from each other equidistant from the center.

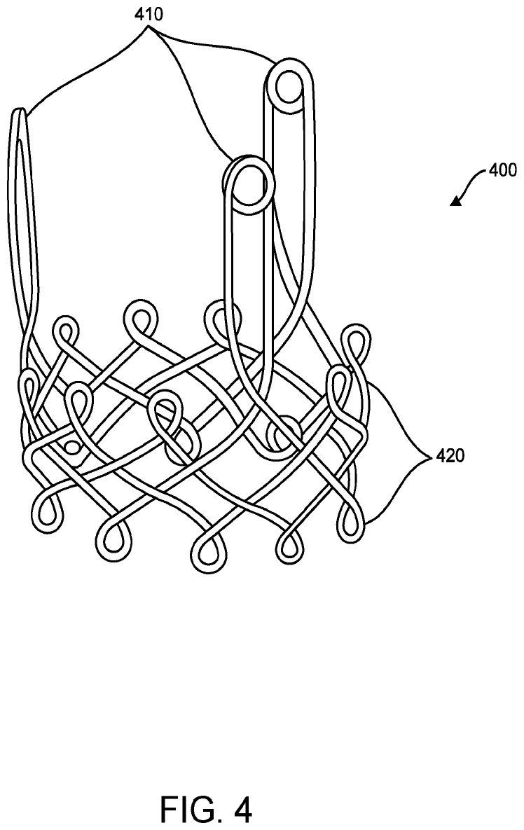

FIG. 4 generally illustrates an embodiment of a tubular, braided frame as disclosed herein. As shown in FIG. 4, a braided frame 400 may comprise commissure posts 410 and a sealing zone 420, wherein the braided frame 400 has a braided tube length that is shortened considerably when compared to other embodiments disclosed herein that still provides a minimal frame necessary for connection to leaflet structure.

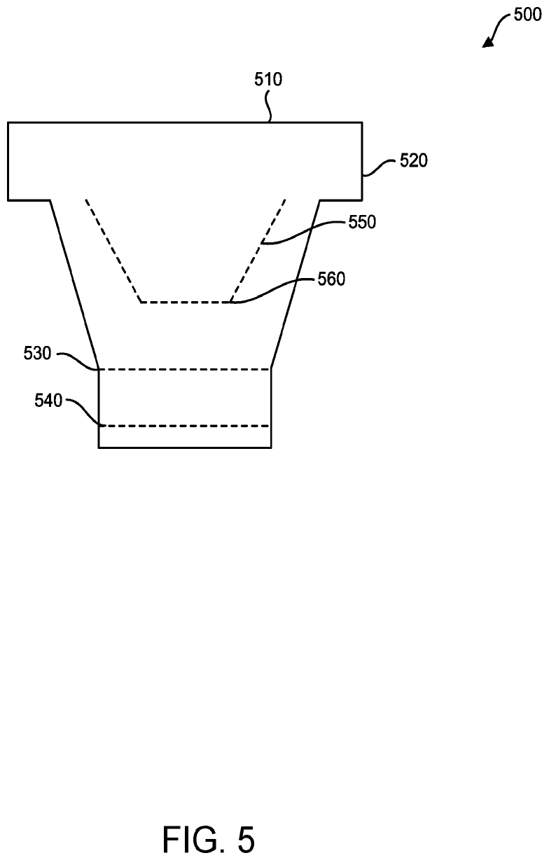

FIG. 5 generally illustrates an embodiment of a leaflet panel as disclosed herein. A leaflet panel may be defined as a pattern, cut from synthetic or biological material, that serves as a single leaflet. For example, excised porcine or bovine pericardium may be used for a leaflet panel. The combination and attachment of two or more leaflet panels create a leaflet assembly, such as a Z-valve insert. As shown in FIG. 5, a leaflet panel 500 may comprise a distal end 510 that when incorporated into a completed valve, becomes the co-apting closing zone of the valve, where the three proximal ends are forced together in a Y form, called a co-apt, to close the valve. The outer ends 520 of the distal end 510 have tabs that are used to sew the leaflets to the commissure posts. Towards the proximal end, a base stitch zone 530 is used to create a sealing zone, by itself or in combination with an inflow stitch zone 540. An inflow stitch 540 is generally parallel to and on the inflow side of the base stitch and serves to attach the Z-valve insert to a frame or cuff. The material between the inflow stitch and the base stitch can be a continuous part of the Z-valve insert, or be a different material sewn to the Z-valve insert.

A belly stitch is defined as a stitch originating at the edge seams of the Z-valve insert and following a wire to define an edge of a leaflet, with the option to attach to one of the wires of the frame and/or cuff. The wire where the belly stitch is attached may be shaped-set to further improve leaflet durability and performance. A cuff may be defined as additional material positioned either on the outside or inside of the frame and may be extended along the top and bottom of the frame, though at a minimum is attached above and below the base stitch. The belly stitch serves the purpose of improving leaflet durability and hemodynamic performance. A valve belly stitch 550 is angled from the distal outer sections towards the middle center of the leaflet and may, in one embodiment, either be sewn to the braided frame or an outer cuff. A bellows portion 560 creates the bottom of the belly and may or may not be sewn to the frame. A bellows portion 560 of the belly stitch 550 may be defined as an interruption of attachment or following of the belly stitch, generally at the center of the leaflet, that serves to improve collapsibility.

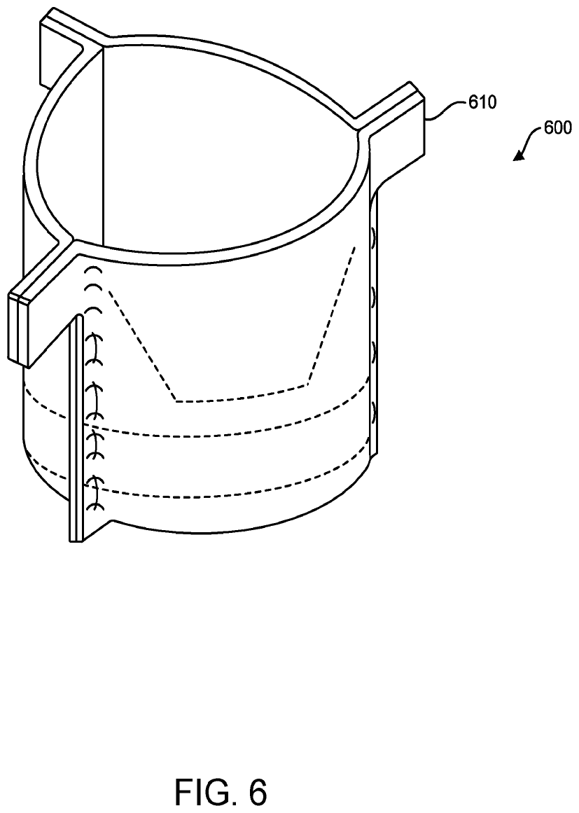

FIG. 6 generally illustrates an embodiment of a Z-valve insert as disclosed herein. As shown in FIG. 6, a Z-valve insert 600 may comprise three leaflet panels, with commissure ends 610 of each leaflet panel connected and stitched together along the ends to create a tube-like, cylindrical structure. The edges may be parallel or have a specified angle, so as to optimize durability and hemodynamic performance. A Z-valve insert 600 may also comprise an optional tubular portion on the inflow side of the base stitch, created by overlapping the lower tabs of the Z-valve insert 600. In one embodiment, the Z-valve insert 600 is attached to the commissure posts of a tubular frame via a base stitch. The base stitch may be located at the top, middle, or along the bottom of the tubular frame. In another embodiment, the base and inflow stitch lines may pass through the leaflets, the braid, and the cuff, wherein the region on the outside of the valve frame between the base stitch and the inflow stitch creates a sealing zone.

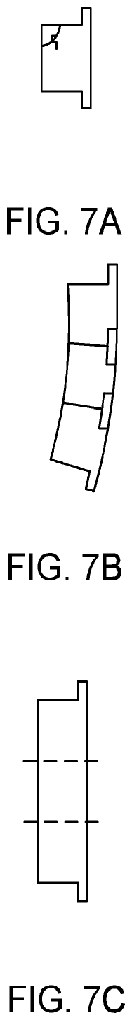

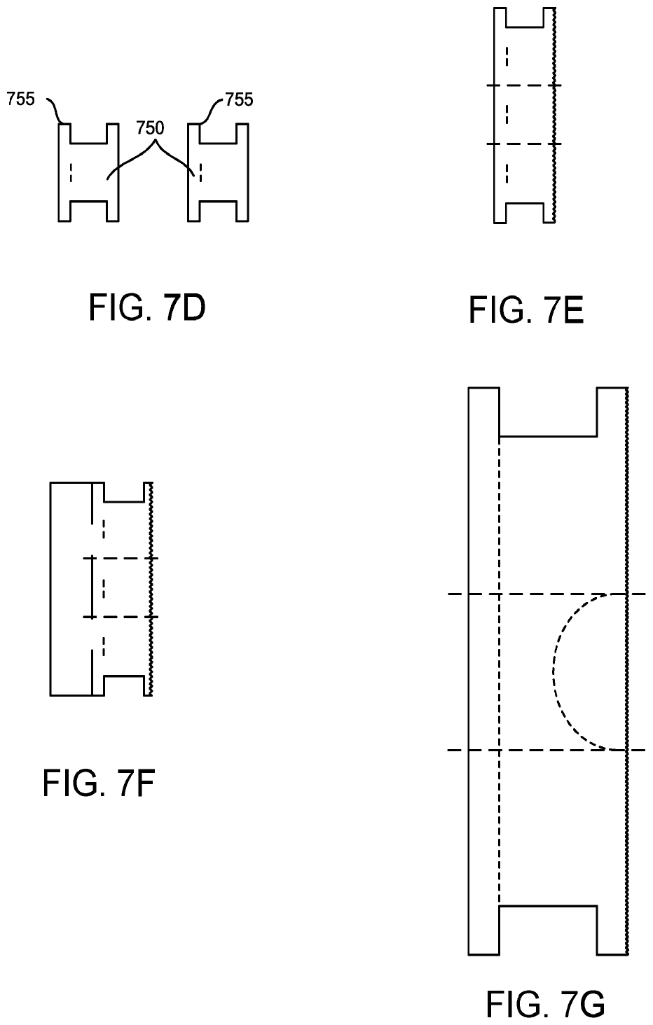

FIGS. 7A-7G generally illustrate embodiments of patterns for leaflet panels. The embodiment in FIG. 7A discloses a view of the leaflet assembly 500. FIG. 7B discloses a pattern combining three separate leaflets. FIG. 7C is a variation of the assembly showing a straight edge which allows for attachment of additional cuff material.

FIG. 7D discloses a preferred embodiment of a valve leaflet pattern 750 with a cuff 755 at the proximal end. The cuff 755 may be used to wrap over the proximal end of a frame and create an outer sealing zone in addition to the inner sealing zone. FIG. 7E shows three leaflets combined and incorporating a cuff. FIG. 7F discloses the combination of 7E incorporating a larger cuff, which can serve to seal the complete outer frame spanning the length of a valve assembly. FIG. 7G discloses a scale version of a combined 3-piece leaflet.

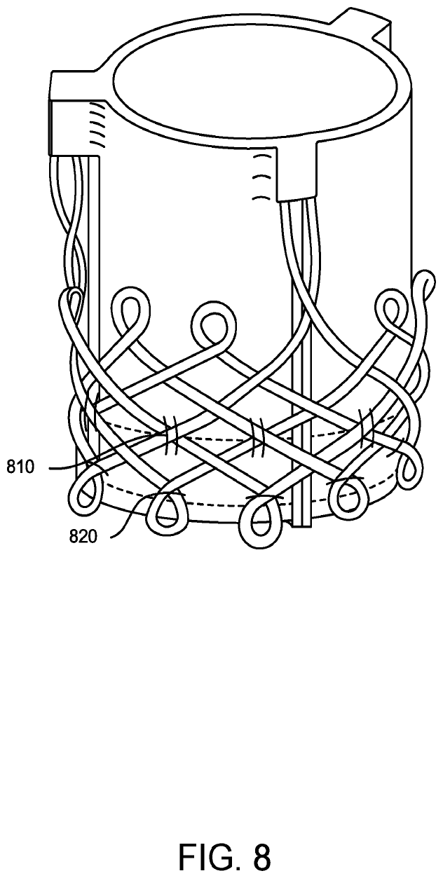

FIG. 8 generally illustrates an embodiment of a collapsible, heart-valve assembly system as disclosed herein. The valve assembly in FIG. 8 comprises an extended sealing zone created by a longer leaflet that is sewn to the structure at the centerline 160 and baseline stitch, and also at the proximal braid end along inflow stitch line 810.

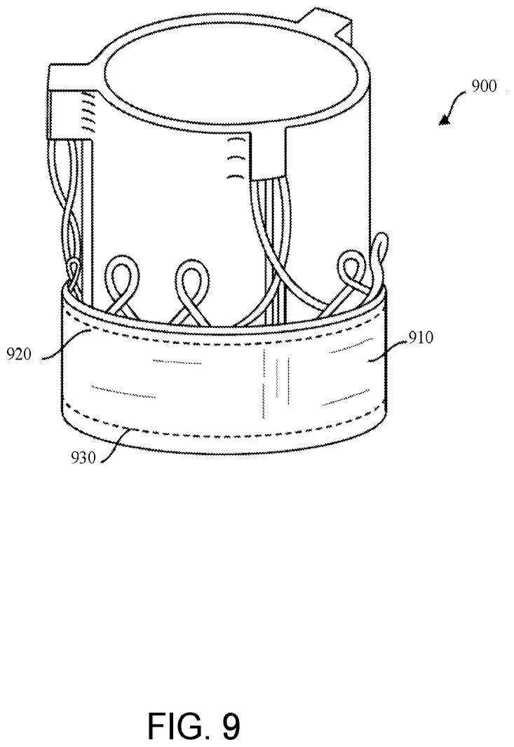

FIG. 9 generally illustrates an embodiment of a collapsible, heart-valve assembly system as disclosed herein. As shown in FIG. 9, the valve assembly 900 may comprise—in addition to the tubular, braided frame 110 and leaflet structure 120 incorporated into the frame 110—a cuff 910, wherein the cuff 910 covers the proximal portion of the valve assembly 900. A cuff 910 may be defined as material that is attached to the frame 110, wherein the cuff 910 may be positioned either on the outside or inside of the frame and may be extended along the top and bottom of the frame, though at a minimum is attached above and below the base stitch 920. In another embodiment, the cuff 900 may be attached alongside an inflow stitch line 930. The cuff material may be 1) elastic and deform with the braid, 2) non-elastic, wherein the braid wires slide through/around the attached cuff, or 3) a combination of both. Cuff may also comprise a polymeric coating (e.g. chronosil) or a continuous knit, woven or braided fabric. And cuffs may be rolled up with a seam or a tubular structure.

In another embodiment, a continuous cuff may be sewn to the Z-valve insert at the baseline stitch location and wrapped around the inflow edge of the braided valve frame to become a cuff on the outer side of the frame. And in a separate embodiment, a valve may have both an inner cuff and an outer cuff, and/or partial cuffs that cover discrete portions of the braided valve frame.

Cuffs 910 are generally used for covering the wires of the frame 110 so as to provide a sealing zone, wherein a sealing zone, or ring, is formed to prevent blood flow from either side while the leaflets provide flow control. The sealing zone is comprised of either flexible or non-flexible material. Cuffs 910 also serve the purpose of attaching the Z-valve insert 120 to the frame 110. In a preferred embodiment, the cuff 910 attached along the top and bottom edges of the frame 110, or along a row of crossing points. A cuff 910 may be attached to the frame 110 along all adjacent wires, such as with a stitch that does not interfere with the motion of the braid crossing points.

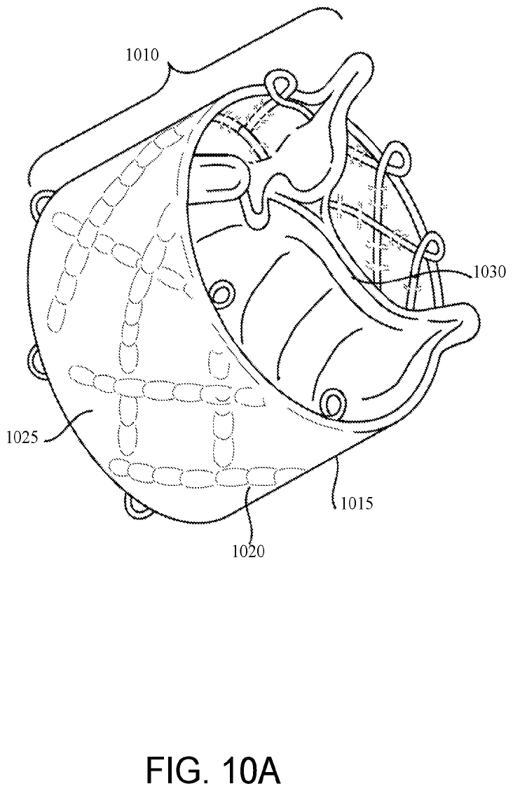

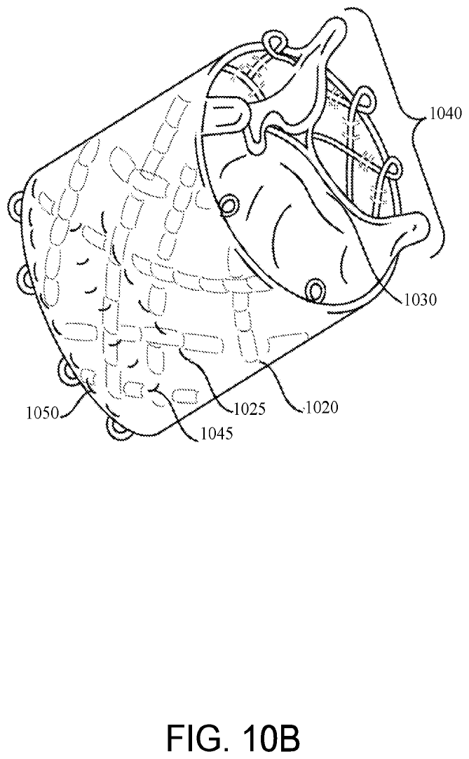

FIG. 10A generally illustrates an embodiment of a collapsible, heart-valve assembly system as disclosed herein. As shown in FIG. 10A, in one embodiment, a valve assembly 1010 comprises a braided frame similar to FIG. 2, and a long cuff covering 1015 on the outside. This cuff over the complete outer frame may serve as an extended sealing zone. A belly stitch 1020 may be sewn to the frame whereas a bellows stitch 1025 is not sewn to the frame. In this embodiment, the distal leaflet ends 1030 are shown co-apting so as to close the valve in a loose Y-shape. In some embodiments, the valve co-apt area may comprise some “looseness” so as to ensure sufficient and effective contact among all three leaflets and ensure complete closing of the valve. Leaflets may be constructed of tissue such as porcine pericardium or other materials known to the art. In some cases, valves or parts of valves excised from animals may be sewn into the disclosed frame structure.

FIG. 10B generally illustrates an embodiment of a collapsible, heart-valve assembly system as disclosed herein. As shown in FIG. 10B, a valve assembly may comprise combined commissure posts and leaftlet tabs 1040, a leaflet end, co-apt zone 1030, a belly stitch 1020, a bellows stitch 1025, a base stitch 1045, and an inflow stitch 1050.

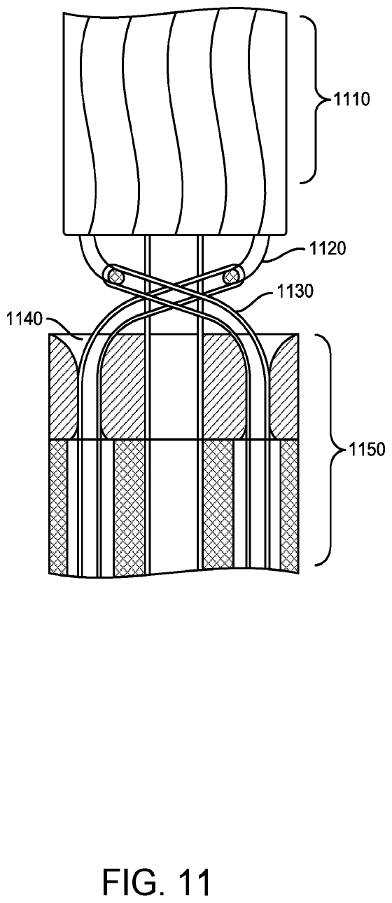

FIG. 11 generally illustrates an embodiment of a delivery system for a collapsible, heart-valve assembly as disclosed herein. Preferably a proximal, or in-flow side, delivery system. As shown in FIG. 11, a delivery system may comprise a compressed valve 1110 and loops 1120 that are slidably retained by suture lines 1130. The suture lines 1130 follow a pathway through bushings 1140 incorporated into the delivery catheter 1150. The sutures 1130 may control the expansion and retention of the valve assembly as desired.

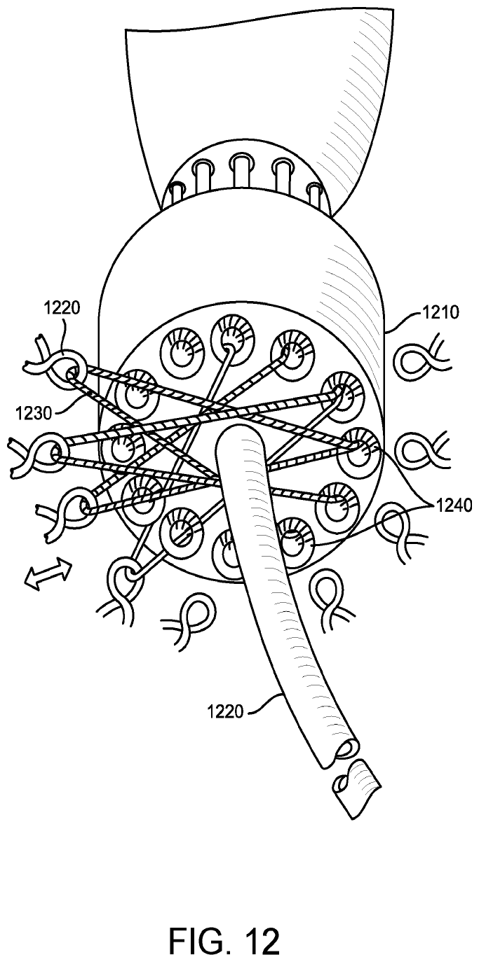

FIG. 12 generally illustrates an embodiment of a delivery system as described in the previous FIG. 11 for a collapsible, heart-valve assembly as disclosed herein. As shown in FIG. 12, each loop 1220 has a suture 1230 threaded through it, wherein the sutures 1230 exit from and return to bushings 1240 on a manifold 1210. The pattern of the sutures 1230 alternates in direction—starting from a bushing on the opposite side of a loop, through the loop, and then back to a bushing, such that the sutures create tension with a vector through the centerline. This has the benefit of providing the aforementioned centerline tension vector for each frame loop, while avoiding the tubing that extends through the center of the delivery catheter. This pattern also has the benefit of providing tension to draw down on and keep the loops as close to the center as possible. Each suture has an end that is fixed relative to the delivery catheter, and an opposite end which, when properly configured in length, can be pulled or released in concert with the rest of the sutures to precisely control the expansion of the valve. When the valve is to be released, the sutures can be cut/released and withdrawn. This mechanism can be used to preferentially control the expansion of the proximal valve' but could be used to control the distal valve as well.

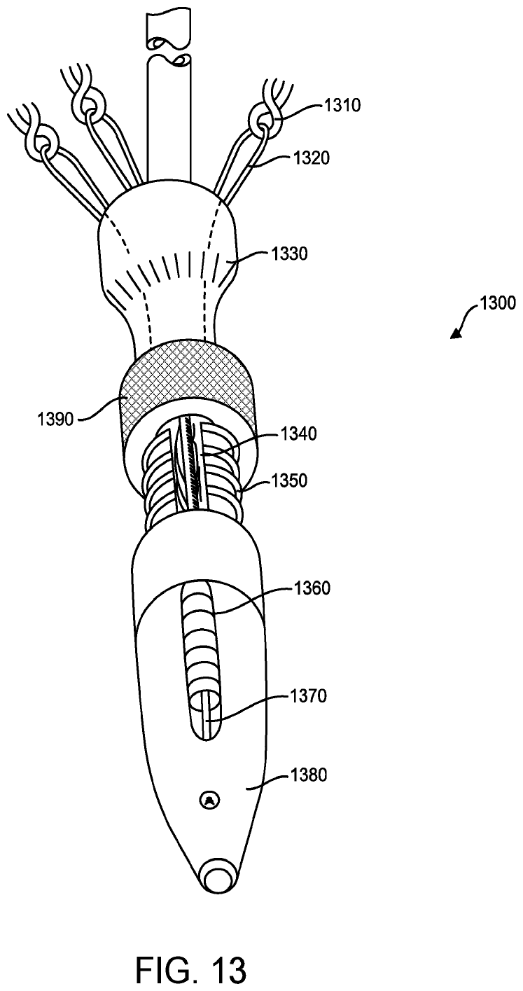

FIG. 13 generally illustrates an embodiment of a delivery system of a collapsible, heart-valve assembly as disclosed herein. As shown in FIG. 13, a delivery system 1300 for control of a distal valve may comprise distal valve loops 1310 that are slidably connected to short-length sutures 1320. The sutures 1320 are guided through a funnel bushing 1330, through a channel 1340 in a threaded rod 1350, and terminated with loops 1360 on a wire 1370, which is slidably retained in distal tip 1380. The wire 1370 is accessible at the proximal end of the delivery system. A knob 1390 is turned to push the bushing 1330 prior to delivery to pull the loops 1310 to a compressed position. The valve is released by pulling the wire 1370, releasing one end of the sutures 1320.

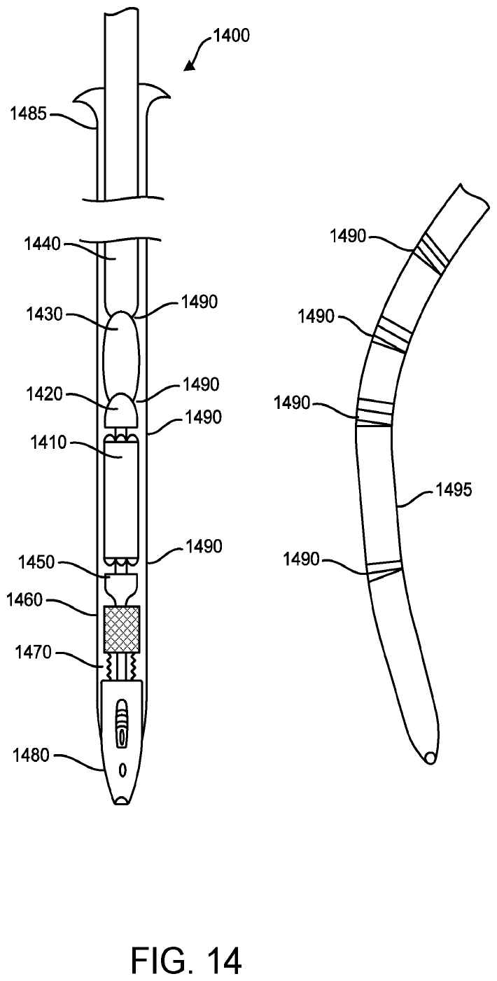

FIG. 14 generally illustrates an embodiment of a delivery system of a collapsible, heart-valve assembly as disclosed herein. FIG. 14 discloses a distal cut-out, side view 1400 and a distal outside-view 1495 of the combined assemblies described in drawings 11-13, along with additional structures. FIG. 14 discloses a valve 1410 in a compressed form, with a proximal release-mechanism manifold 1420. The manifold 1420 may be abutted to a bearing 1430, that allows some angular articulation between it and the manifold 1420 and possibly another bushing 1440. These bushings can provide some angular articulation while allowing the sutures and center tubing through them. The release mechanism for the distal valve comprises a funnel bushing 1450, a knob 1460, a threaded rod 1470, and a distal tip 1480. Also shown is an outer sheath 1485, which is pushed over the assembly prior to delivery to ensure complete compression of the valve and smooth outer surface for insertion in the body. This sheath may then be retracted proximally from outside the body to expose the valve and release mechanisms. Angular articulation zones are shown at points 1490, where some amount of flexibility is gained by the structure design. In some embodiments, the sheath 1485 may also be configured like a jacket with a separable seam running parallel to the central axis of the sheath 1485, the seam being connected to a pull wire such that the seam separates when the wire is pulled proximally from a position outside the body, such that release of the jacket seam allows the valve to expand.

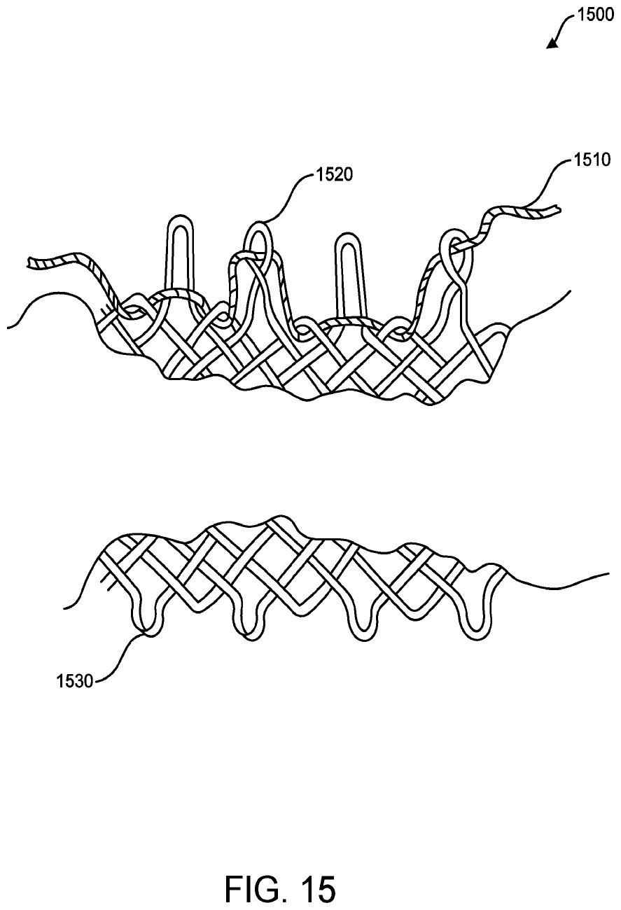

FIG. 15 generally illustrates an embodiment of a retrieval system for a collapsible, heart-valve assembly as disclosed herein. FIG. 15 discloses an embodiment of a retrieval system 1500 for the removal of a valve from the cardiac structure and receiver after delivery. The retrieval system 1500 may comprise a leash 1510, which may be permanently incorporated into the loops 1520 of the braided frame. In some embodiments, the leash 1510 may be comprised of radiopaque material for visibility under fluoroscopy. The leash 1510 may be captured by one or more retrieval hooks on a catheter that may then be pulled into the catheter or a specifically designed retriever. The tension of the leash 1510 and hooks may partially compress the valve, separating it from the receiver.

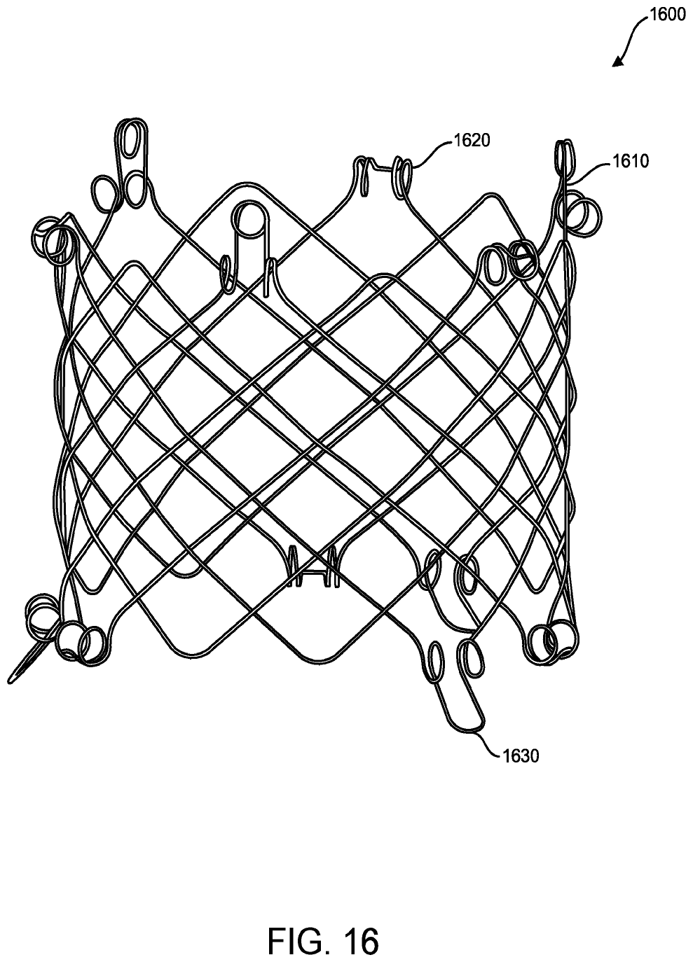

FIG. 16 generally illustrates an embodiment of a braided frame of a collapsible, heart-valve assembly system as disclosed herein. FIG. 16 discloses a braided frame 1600 that comprises additional features, such as commissures 1610 with wire coils 1620 and 1630 that have an axis parallel to the tangent of the frame circumference. Coils 1620 and 1630 may act as springs to increase the strength of the commissures 1610, which in turn provides resistance to flow forces during valve closure. Coils may be designed using parameters such as wire diameter, loop coil diameter, and coil turns to optimize valve performance. Further, coils 1620 and 1630 may comprise variations designed to create a latch for releasably joining the frame to a receiver. The round nature of the coils creates a spring-like latch for engaging a receiving geometry such as a cylinder or a custom, even asymmetrical, shape The extended latch-feature of coil 1630 may be used in concert with a leash 1510, which would pull in on the latch coil 1630, towards the center of the valve axis, bending the latch coil 1630 to release from a receiver.

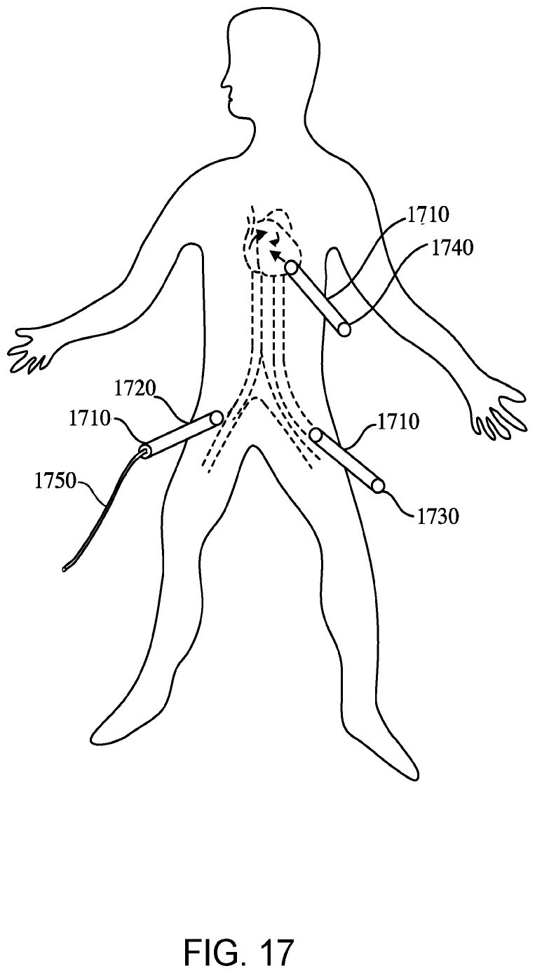

FIG. 17 generally illustrates an embodiment of a deployment system for a collapsible, heart-valve assembly system as disclosed herein. As shown in FIG. 17, several methods are available for deployment of the components of the replacement heart valve system to a desired target cardiac structure. FIG. 17 depicts at least three different pathways suitable to deliver the components to a mitral valve structure. A “transapical” approach comprises inserting the guiding catheter 1710 in the groin 1720 into a vein and up to the mitral valve via the atrial septum. A “transaortic” approach comprises inserting the guiding catheter 1710 into the groin 1730 into an artery and then up to the mitral valve via the aortic valve. An alternative “transapical” approach 1740 comprises surgically exposing the heart and inserting the guiding catheter 1710 into the apex of the target heart. Methods for delivering the heart-valve assembly system may also include the use of a guidewire 1750, wherein the heart-valve assembly system is inserted into a vein over a guidewire.

Other embodiments may include combinations and sub-combinations of features described or shown in the several figures, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

References throughout this specification to “one embodiment,” “an embodiment,” “an example embodiment,” etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with one embodiment, it will be within the knowledge of one skilled in the art to affect such feature, structure, or characteristic in connection with other embodiments whether or not explicitly described.

Unless the context clearly indicates otherwise (1) the word “and” indicates the conjunctive; (2) the word “or” indicates the disjunctive; (3) when the article is phrased in the disjunctive, followed by the words “or both,” both the conjunctive and disjunctive are intended; and (4) the word “and” or “or” between the last two items in a series applies to the entire series.

Where a group is expressed using the term “one or more” followed by a plural noun, any further use of that noun to refer to one or more members of the group shall indicate both the singular and the plural form of the noun. For example, a group expressed as having “one or more members” followed by a reference to “the members” of the group shall mean “the member” if there is only one member of the group.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

What is claimed is:

1. A replacement heart-valve delivery system, comprising:
a replacement heart valve, comprising a tubular braided frame, a leaflet assembly, and one or more commissure tabs;
wherein the tubular braided frame comprises an inflow end, an outflow end, and one or more looping structures on one or both of the inflow end and the outflow end, and
wherein the leaflet assembly comprises at least one valve leaflet, wherein the at least one leaflet comprises an inflow end, an outflow end, and one or more commissure tabs extending horizontally away from the inflow end and connecting to the inflow end of the tubular braided frame;
a delivery component, comprising one or more suture lines and one or more bushings connected to a channel retained in a threaded rod;
wherein the one or more suture lines goes through the one or more looping structures, through the one or more bushings, through the channel in the threaded rod, and connects to a controlling mechanism.

2. The system of claim 1, wherein the tubular braided frame is a braid of one or more wires, wherein the braid of the one or more wires is either a zig-zag braid or an over-under braid, and wherein the one or more wires consists of one of: nitinol wire, stainless steel, cobalt chrome, and nylon.

3. The system of claim 2, wherein the controlling mechanism comprises a linking element for connecting the one or more suture lines to a knob.

4. The system of claim 3, wherein the knob controls the one or more suture lines and movement of the one or more looping structures.

5. The system of claim 2, wherein the one or more suture lines alternate in direction starting at a first bushing of the one or more bushings, through the one or more looping structures, and ending at a second bushing of the one or more bushings, such that the one or more suture lines creates tension with a vector extending through a centerline of the delivery component.

6. The system of claim 2, further comprising a retrieval system, wherein the retrieval system comprises a leash incorporated into the one or more looping structures.

7. The system of claim 2, wherein the leaflet assembly comprises two or more valve leaflets, wherein the one or more commissure tabs of the two or more leaflets are connected and wherein the outflow end of the two or more valve leaflets are connected to form a y-shape.

8. The system of claim 7, wherein the connected one or more commissure tabs and the connected outflow end of the two or more valve leaflets are connected by one or more of: sewing, fusing, and stitching

9. A method for percutaneous deployment and placement of a replacement heart-valve delivery system, comprising:
the replacement heart-valve delivery system comprising a replacement heart valve and a delivery component;
wherein the replacement heart valve comprises a tubular braided frame, a leaflet assembly, and one or more commissure tabs;
wherein the tubular braided frame comprises an inflow end, an outflow end, and one or more looping structures on one or both of the inflow end and the outflow end; and
wherein the leaflet assembly comprises at least one valve leaflet, wherein the at least one leaflet comprises an inflow end, an outflow end, and one or more commissure tabs extending horizontally away from the inflow end and connecting to the inflow end of the tubular braided frame;
wherein the delivery component comprises one or more suture lines, a release wire, and one or more bushings connected to a channel retained in a threaded rod;
wherein the one or more suture lines goes through the one or more looping structures of the replacement heart valve, through the one or more bushings, through the channel in the threaded rod, connects to the release wire, and connects to a controlling mechanism, wherein the controlling mechanism comprises a linking element for connecting the one or more suture lines to a knob;
wherein pulling the one or more suture lines with the knob controls the expansion of the one or more looping structures and the expansion of the replacement heart valve, and wherein pulling the release wire releases the suture lines and releases the replacement heart valve;
percutaneously placing the replacement heart-valve delivery system into one of a vein or an artery;
delivering the replacement heart-valve delivery system to a native heart valve;
placing the replacement heart-valve delivery system in the position of the native heart valve;
pulling the one or more suture lines to expand the replacement heart valve; and
pulling the release wire to release the replacement heart valve.

10. The method of claim 9, wherein the vein is a femoral vein and wherein delivering the replacement heart-valve delivery system to a native heart valve comprises delivering the replacement heart-valve delivery system through the vena cava.

11. The method of claim 9, wherein the vein is a femoral vein and wherein delivering the replacement heart-valve delivery system to a native heart valve comprises delivering the replacement heart-valve delivery system through the vena cava and through a puncture in the atrial septum.

12. The method of claim 9, wherein the tubular braided frame is a braid of one or more wires, wherein the braid of the one or more wires is either a zig-zag braid or an over-under braid, and wherein the one or more wires consists of one of: nitinol wire, stainless steel, cobalt chrome, and nylon.

13. The method of claim 9, wherein the one or more suture lines alternate in direction starting at a first bushing of the one or more bushings, through the one or more looping structures, and ending at a second bushing of the one or more bushings;
The method further comprising, controlling the one or more suture lines with the knob to create tension with a vector extending through a centerline of the delivery component.

14. The method of claim 9, further comprising a retrieval system, wherein the retrieval system comprises a leash incorporated into the one or more looping structures.

15. The method of claim 9, wherein the leaflet assembly comprises two or more valve leaflets, wherein the one or more commissure tabs of the two or more leaflets are connected and wherein the outflow end of the two or more valve leaflets are connected to form a y-shape.

16. The method of claim 15, wherein the connected one or more commissure tabs and the connected outflow end of the two or more valve leaflets are connected by one or more of: sewing, fusing, and stitching.

17. The method of claim 9, wherein percutaneously placing the replacement heart-valve delivery system into one of a vein or an artery consists of inserting the replacement heart-valve delivery system into one of the vein or the artery over a guidewire.

18. The method of claim 9, wherein the delivery component further comprises an outer sheath and the placing of the replacement heart-valve delivery system in the position of the native heart valve further comprises releasing the outer sheath to allow the replacement heart valve to expand.
